(19) United States
(12) Patent Application Publication
(10) Pub. No.: US 2004/0115822 A1
(43) Pub. Date: Jun. 17, 2004

(54) Methods of measuring the dissolution rate of an analyte in a non-aqueous liquid composition

(76) Inventors: Randal Lee Schapaugh, Battle Creek, MI (US); Jamie K. North, Vicksburg, MI (US); Edward L. Ciolkowski, Galesburg, MI (US); Leslie C. Eaton, Schoolcraft, MI (US)

Correspondence Address: PHARMACIA & UPJOHN, 301 HENRIETTA ST, 0228-32-LAW, KALAMAZOO, MI, 49007, US

(21) Appl. No.: 10/658,165

(22) Filed: Sep. 9, 2003

Related U.S. Application Data
(60) Provisional application No. 60/410,147, filed on Sep. 12, 2002.

(51) Int. Cl. G01N 33/00

(52) U.S. Cl. 436/91

(57) ABSTRACT

The present invention provides a method of characterizing the transfer of an analyte from a non-aqueous liquid composition to an aqueous medium and in particular to an in vitro method for measuring the dissolution of a drug from a sustained release dosage form.

### CROSS REFERENCE TO RELATED APPLICATION

This application claims the benefit of U.S. Provisional Application Serial No. 60/410,147, filed Sep. 12, 2002, under 35 USC 119(e)(i), which is incorporated herein by reference in its entirety.

### FIELD OF THE INVENTION

The present invention refers to a method of characterizing the transfer of an analyte from a non-aqueous liquid to an aqueous medium and in particular to an in vitro method for measuring the dissolution of a drug from a sustained release dosage form.

### BACKGROUND OF THE INVENTION

One important aspect of formulating pharmaceutical compositions is the drug's pharmacokinetic behavior. Depending on a variety of factors, such as the physical state of the drug (i.e. gas, liquid, solid), its crystal form, its particle size, the dosage form, and the excipients used, the time-dependent release of the drug in the body can vary drastically. Even if the same drug is presented in the same dosage form lot-to-lot variations can occur.

For regulatory approval pharmacokinetic behavior is often determined by administering the drug to animals or humans and measuring the amount of drug or its metabolites e.g. in blood at certain points of time after administration. This method is time-consuming and expensive and is generally not employed to control the quality of the pharmaceuticals during the manufacturing process. A number of methods have been devised to assess the in vivo pharmacokinetic behavior of drugs in in vitro tests. Some of the tests have been standardized and are described e.g. in the United States Pharmacopeia (USP). Commonly used USP methods are the basket method (USP method I) and the paddle method (USP method II). In addition to these standardized methods, a large number of methods for specific individual applications have been described. An overview over a number of dissolution methods can be found e.g. in G. K. Shiu, Drug Information Journal, 30, 1045-1054, (1996).

Andonaegui et al. (Drug Development and Industrial Pharmacy, 25(11), 1199-1203 (1999)) describe an in vitro method for predicting the in vivo performance of sustained-release theophylline matrix tablets administered in fasted conditions and with a high-fat diet. The dissolution profiles of theophylline in three types of sustained-release matrix tablets were investigated. To improve the in vitro/in vivo-correlation for a high-fat diet the tablets were pretreated by mixing with peanut oil before the dissolution testing in the USP paddle test.

Japanese patent application JP 05-249097 describes a dissolution test for predicting the in vivo release of a sustained-release tablet. The tablet is subjected to the paddle method, taken out, treated with oils and fats and then either returned to the paddle apparatus together with beads in the aqueous dissolution medium or submerged in a basket. This method is said to predict the concentration of a drug in blood plasma inside a living body without being affected by the release control mechanism of the sustained release tablet.

U.S. Pat. No. 6,132,751 discloses the evaluation of solubitity of drug in an ophthalmic emulsion composition in PBS.

Various in vitro dissolution methods for microparticulate drug delivery systems are compared by Conti et al. in Drug Development and Industrial Pharmacy, 21(10), 1233-1233 (1995). The influences of stirring speed, ionic strength and the presence of a surfactant are investigated.

Dissolution methods for testing oily drug preparations have also been described. Takahashi et al. (Chem. Pharm. Bull., 42(8), 1672-1675, (1994)) compare the paddle method and the rotating dialysis cell method. In a variation of the rotating dialysis cell method octanol was employed as external phase, while an acidic solution was used as an internal phase.

Machida et al. (Chem. Pharm. Bull., 34(6), 2637-2641, (1986)) describe one attempt to overcome the problems encountered in measuring the dissolution characteristics of oily drug preparations. They propose using a modification of the paddle method of the Japanese Pharmacopeia method 2 with an additional assistant wing to stir the surface of the aqueous dissolution medium. Furthermore, beads were added to improve agitation and a bile salts solution was employed as the aqueous dissolution medium.

The pharmacokinetic behavior of non-aqueous pharmaceutical compositions, in which the drug is dissolved, dispersed, suspended, or otherwise provided, in a non-aqueous base, is difficult to predict reliably using prior art methods. The precision and reliability of the in vitro measurements is often low and the results of the in vitro measurements do not always correlate with the behavior of the drug in vivo. Therefore, one object of the present invention is to provide a reliable method, with which to characterize the dissolution of an analyte in a non-aqueous liquid composition. A further object of the present invention is to provide a method with which to characterize the dissolution of an analyte having a low solubility. Another object of the invention is to provide a method with which to characterize the dissolution of analytes which are only slowly dissolved. Yet another object of the invention is to provide a rapid method with which to characterize the dissolution of analytes which can be utilized during manufacture as an in-process assay. Other objects of the invention will be readily apparent from reading the specification and claims of the application.

### BRIEF DESCRIPTION OF THE FIGURES

FIG. 1 shows one possibility of plotting the dissolution rate if the amount of analyte is determined more than once.

FIG. 2 shows a further possibility of plotting the dissolution rate if the amount of analyte is determined more than once.

FIG. 3 shows a scheme of a shaker.

FIG. 4 shows the results of the dissolution test of Example 1 using Tween 80 as a surfactant.

FIG. 5 shows the results of the dissolution test of Example 1 using Tween 20 as a surfactant.

FIG. 6 shows the results of the in vitro-in vivo correlation of Example 2.

### SUMMARY OF THE INVENTION

The present invention refers to a method of characterizing the dissolution of an analyte in a non-aqueous liquid composition, comprising the steps of:

(a) providing a non-aqueous liquid composition comprising an analyte and a non-aqueous base;

(b) combining the non-aqueous liquid composition with an aqueous dissolution medium;

(c) agitating the non-aqueous liquid composition and the aqueous dissolution medium to form an emulsion; and

(d) determining the amount of analyte in the aqueous dissolution medium.

### DETAILED DESCRIPTION OF THE INVENTION

The present invention provides a reliable method for characterizing the in vitro dissolution of an analyte in a non-aqueous liquid composition. Although the method is preferably employed to quantitate the dissolution of a pharmaceutically active ingredient from a pharmaceutical composition, it can also be employed in other fields of analytical chemistry, e.g. to determine the rate with which contaminants are leached from oils into the environment, to determine the rate with which active agents such as corrosion inhibitors and the like are depleted from oily bases or to measure the rate with which components are released from pesticides or fertilizers.

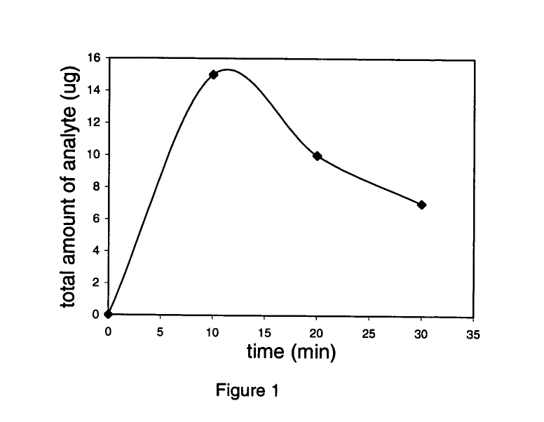

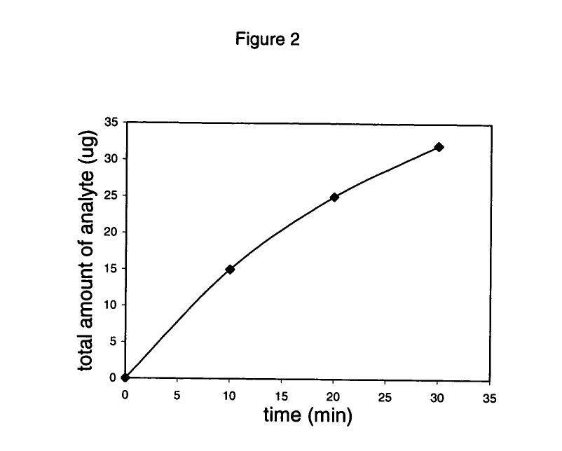

Dissolution may be characterized and quantitated in a variety of ways. For instance, the amount of analyte in the aqueous dissolution medium might only be determined at one predetermined time. For example, if it is determined that 3 μg of analyte have been dissolved after 30 minutes, the dissolution could be characterized as 3 μg dissolved in 30 min. If the amount of analyte in the aqueous dissolution medium is determined more than one time, then the dissolution can be illustrated in several different ways, which are known in the art. One common way is to plot the data in a two-dimensional graph. The x-axis represents the time line. The y-axis represents the amount of analyte dissolved between the nth and the (n−1)th analysis of the aqueous dissolution medium. A further common way is to plot the data in a two-dimensional graph, in which the x-axis is again a time line. The y-axis represents the total amount of analyte dissolved between the beginning of the measurement and the nth analysis of the aqueous dissolution medium. Of course the same information can be presented in a table or any other suitable form other than the two-dimensional graphs discussed above. The following series of experiments can be used as an example: a non-aqueous liquid composition is investigated and the amount of analyte dissolved is determined at 10 minutes (n=1), 20 minutes (n=2), and 30 minutes (n=3). After 10 minutes 15 μg of analyte have dissolved, after 20 minutes 25 μg of analyte have dissolved and after 30 minutes 32 μg of analyte, in total, have dissolved. In the first case the plot as shown in FIG. 1 would be obtained, while in the second case the plot would be as in FIG. 2.

As used herein the term “non-aqueous liquid composition” is any composition which is liquid at the contacting temperature and which comprises an analyte and a non-aqueous base. The mixture of the analyte and the non-aqueous base can be in any form, such as a solution, an emulsion or suspension. If the analyte is suspended in the non-aqueous base, the particle size of the analyte will generally be in the range of from about 50 nm to about 200 microns, preferably from about 100 nm to about 200 microns. The concentration of the analyte in the non-aqueous liquid composition is not particularly restricted. It can, for example, vary from about 0.00001 mg/ml to about 5,000 mg/ml, preferably from about 0.01 mg/ml to about 1,000 mg/ml.

The non-aqueous liquid composition is preferably a pharmaceutical composition. In the methods of the present invention the pharmaceutical composition will generally be a liquid suitable for parenteral, oral, sublingual, intranasal, intrabronchial, pulmonary, intramammary, rectal, vaginal, ocular, or topical application. However, it is also possible to determine the dissolution rate of an analyte in a pharmaceutical composition where the pharmaceutical composition is contained in a capsule. In this case, the shell of the capsule will disintegrate on contact with the aqueous dissolution medium and release its contents.

The analyte can be any component of the non-aqueous liquid composition, the dissolution of which is to be characterized. Examples of analytes are, but are not restricted to, a contaminant, an active component, or an inactive component. In the case of pharmaceutical compositions the analyte will typically be the pharmaceutically active ingredient; but it can also be an excipient or any other component of the pharmaceutical composition. The method of the present invention is not restricted to the determination of a single analyte; if desired two or more analytes can be determined. The method of the invention is not restricted to the determination of analytes with any particular physical or chemical characteristics. Virtually any analyte—organic or inorganic—can be determined with the method of the invention so long as the analyte is at least partially soluble in the aqueous dissolution medium chosen for the method. Examples of analytes, which can be determined using the method of the invention include the following illustrative, non-limiting classes: ACE inhibitors; α-adrenergic agonists; β-adrenergic agonists; α-adrenergic blockers; β-adrenergic blockers (beta blockers); alcohol deterrents; aldose reductase inhibitors; aldosterone antagonists; amino acids; anabolics; analgesics (both narcotic and non-narcotic); anesthetics; anorexics; antacids; anthelmintics; antiacne agents; antiallergics; antiandrogens; antianginal agents; antianxiety agents; antiarrythmics; antiasthmatics; antibacterial agents and antibiotics; antialopecia and antibaldness agents; antiamebics; antibodies; anticholinergic drugs; anticoagulants and blood thinners; anticolitis drugs; anticonvulsants; anticystitis drugs; antidepressants; antidiabetic agents; antidiarrheals; antidiuretics; antidotes; antiemetics; antiestrogens; antiflatulents; antifungal agents; antigens; antiglaucoma agents; antihistaminics; antihyperactives; antihyperlipoproteinemics; antihypertensives; antihyperthyroid agents; antihypotensives; antihypothyroid agents; anti-infectives; anti-inflammatories (both steroidal and nonsteroidal); antimalarial agents; antimigraine agents; antineoplastics; antiobesity agents; antiparkinsonian agents and antidyskinetics; antipneumonia agents; antiprotozoal agents; antipruritics; antipsoriatics; antipsychotics; antipyretics; antirheumatics; antisecretory agents; anti-shock medications; antispasmodics; antithrombotics; antitumor agents; antitussives; antiulceratives; antiviral agents; anxiolytics; bactericidins; bone densifiers; bronchodilators; calcium channel blockers; carbonic anhydrase inhibitors; cardiotonics and heart stimulants; chemotherapeutics; choleretics; cholinergics; chronic fatigue syndrome medications; CNS stimulants; coagulants; contraceptives; cystic fibrosis medications; decongestants; diuretics; dopamine receptor agonists; dopamine receptor antagonists; enzymes; estrogens; expectorants; gastric hyperactivity medications; glucocorticoids; hemostatics; HMG CoA reductase inhibitors; hormones; hypnotics; immunomodulators; immunosuppressants; laxatives; medicaments for oral and periodontal diseases; miotics; monoamine oxidase inhibitors; mucolytics; multiple sclerosis medications; muscle relaxants; mydriatics; narcotic antagonists; NMDA receptor antagonists; oligonucleotides; ophthalmic drugs; oxytocics; peptides, polypeptides and proteins; polysaccharides; progestogens; prostaglandins; protease inhibitors; respiratory stimulants; sedatives; serotonin uptake inhibitors; sex hormones including androgens; smoking cessation drugs; smooth muscle relaxants; smooth muscle stimulants; thrombolytics; tranquilizers; urinary acidifiers; urinary incontinence medications; vasodilators; vasoprotectants; and combinations thereof. It should be understood that any reference herein to a particular drug compound includes tautomers, stereoisomers, enantiomers, salts and prodrugs of that compound and is not specific to any one solid-state form of the drug.

The method of the invention is especially suitable for determining the dissolution rate of cephalosporins such as third generation cephalosporins. Examples thereof are, but are not limited to, ceftiofur, cefepime, cefixime, cefoperazone, cefotaxime, cefpodoxime, ceftazidime, ceftizoxime, ceftriaxone, moxalactam, pharmaceutically acceptable salts and derivatives thereof. A particularly preferred cephalosporin is ceftiofur, pharmaceutically acceptable salts and derivatives thereof.

Ceftiofur is presently commercially available from Pharmacia under the trade designations Naxel® and Excenel®. Another preferred form of ceftiofur is ceftiofur crystalline free acid (CCFA). This compound as well as pharmaceutical formulations thereof are described in U.S. Pat. No. 5,721,359, which is incorporated herein in its entirety.

The non-aqueous liquid composition also contains a non-aqueous base, which is typically liquid at the contacting temperature and may be miscible, partially immiscible, or immiscible with water. The non-aqueous base can be lipid (fats, oils, waxes, sterols, and glycerides), hydrogenated or non-hydrogenated, saturated, unsaturated, or polyunsaturated, and may be further modified by techniques commonly known in the art. The non-aqueous base is preferably selected from the group consisting of natural or synthetic fats, waxes, or oils, more preferably the non-aqueous base is a natural or synthetic oil. The term oil includes triglyceride fats and oils, including those derived from vegetable, animal, mineral, and marine sources. Illustrative examples of synthetic oils suitable as the non-aqueous base include tri-glycerides, or propylene glycol di-esters of saturated or unsaturated fatty acids having from 6 to 24 carbon atoms. Such carboxylic acids are meant to comprise those carboxylic acids having from 6 to 24 carbon atoms such as, for example hexanoic acid, octanoic (caprylic), nonanoic (pelargonic), decanoic (capric), undecanoic, lauric, tridecanoic, tetradecanoic (myristic), pentadecanoic, hexadecanoic (palmitic), heptadecanoic, octadecanoic (stearic), nonadecanoic, eicosanoic, heneicosanoic, docosanoic and lignoceric acid. Examples of unsaturated carboxylic acids include oleic, linoleic, linolenic acid and the like. It is understood that the tri-glyceride vehicle may include the mono-, di-, or triglyceryl ester of the fatty acids or mixed glycerides and/or propylene glycol di-esters wherein at least one molecule of glycerol has been esterified with fatty acids of varying carbon atom length. The following are examples of triglyceryl esters: tri-unsaturated esters including triolein, trilinolein and trilinolenin; saturated tri-saturated esters including tripalmitin, tristearin, and tridecanoin. Further examples of triglyceryl esters include di-saturated-mono-unsaturated types: oleodisaturated esters such as 1,2-dipalmitoyl-3-oleoyl-rac-glycerol or 1,3-dipalmitoyl-2-oleoyl-rac-glycerol; linoleodisaturated esters such as 1,3-dipalmitoyl-2-linoleoyl-rac-glycerol. Further examples of triglycerides are mono-saturated-di-unsaturated esters: such as mono-saturated-oleolinolein esters including 1-Palmitoyl-2-oleoyl-3-linoleoyl-rac-glycerol and 1-linoleoyl-2-oleoyl-3-stearoyl-rac-glycerol, and mono-saturated-dilinolein esters including 1,2-dilinoleoyl-3-palmitoyl-rac-glycerol.

Examples of diglyceril esters include: the di-unsaturated esters such as 1,2-diolein or 1,3-diolein, 1,2-dilinolein or 1,3-dilinolein and 1,2-dilinolenin or 1,3-dilinolenin; saturated di-saturated esters such as 1,2-dipalmitin or 1,3-dipalmitin, 1,2-distearin or 1,3-distearin, and 1,2-didecanoin or 1,3-didecanoin; saturated-unsaturated diglyceril esters such as 1-palmitoyl-2-oleoyl-glycerol or 1-oleoyl-2-palmitoyl-glycerol, 1-palmitoyl-2-linoleoyl-glycerol or 1-linoleoyl-2-palmitoyl-glycerol.

Examples of monoglyceril esters include: unsaturated esters such as 1-olein or 2-olein, 1-linolein or 2-linolein and 1-linolenin or 2-linolenin; saturated esters such as 1-palmitin or 2-palmitin, 1-stearin or 2-stearin, and 1-decanoin or 2-decanoin.

Examples of polyethylene glycol (PEG) di-esters include: di-unsaturated esters such as 1,2-diolein or 1,3-diolein, 1,2-dilinolein or 1,3-dilinolein and 1,2-dilinolenin or 1,3-dilinolenin; saturated di-saturated esters such as 1,2-dipalmitin or 1,3-dipalmitin, 1,2-distearin or 1,3-distearin, and 1,2-didecanoin or 1,3-didecanoin. Further examples of PEG di-esters from saturated-unsaturated diglyceril esters include: 1-palmitoyl-2-oleoyl-glycerol or 1-oleoyl-2-palmitoyl-glycerol, 1-palmitoyl-2-linoleoyl-glycerol or 1-linoleoyl-2-palmitoyl-glycerol.

Illustrative examples of natural oils are canola oil, coconut oil, corn oil, peanut oil, sesame oil, olive oil, palm oil, safflower oil, soybean oil, cottonseed oil, rapeseed oil, sunflower oil and mixtures thereof. Of these cottonseed oil is preferred.

The non-aqueous base may be modified by means known in the art. For example, in embodiments using a peroxidized unsaturated oil base, modified base may have a peroxide value of between about 0.1 and about 600, and in some embodiments about 10, about 20, about 40, or about 80 or any value in between. As used herein, peroxide values are expressed as milliequivalents (mEq) of peroxide per 1000 grams of oil sample.

Apart from the above-mentioned components the non-aqueous liquid composition can also contain additional compounds. For example, if the non-aqueous liquid composition is a pharmaceutical composition, it can contain any pharmaceutically acceptable components. Typical additional components are, for example, pharmaceutically active ingredients, excipients, additives, suspending agents, preservatives, wetting agents, thickeners, buffers and flocculating agents. Suspending agents, such as gums (e.g., acacia, carrageenan, sodium alginate and tragacanth), cellulosics (e.g., sodium carboxymethylcellulose, microcrystalline cellulose, and hydroxyethylcellulose), and clays (e.g., bentonite and colloidal magnesium aluminum) may be included. Preservatives, such as methyl and propyl paraben, benzyl alcohol, chlorobutanol and thimerosal may be added. Anionic surfactants (e.g., docusate sodium and sodium lauryl sulfate), nonionic surfactants (e.g. polysorbates, polyoxamers, octoxynol-9), and cationic surfactants (e.g. trimethyltetradecylammonium bromide, benzalkonium chloride, benzethonium chloride, myristyl gamma picolinium chloride) may be used. Thickeners, such as gelatin, natural gums and cellulose derivatives (such as those listed above as suspending agents) may be added. Buffers, such as citrate and phosphate buffering agents, may be included, as well as osmotic agents, such as sodium chloride and mannitol. For pharmaceutical compositions, which are to be administered orally, flavoring agents, sweeteners (e.g., mannitol, sucrose, sorbitol and dextrose), colorants and fragrances may be employed. In pharmaceutical compositions, excipients such as sorbitan monooleate (available as Span 80® from Sigma-Aldrich) and phosphatidylcholine (available as Phospholipon 90H from American Lecithin Company) may be employed.

The aqueous dissolution medium of the present invention can be any aqueous dissolution medium known in the art. Commonly used dissolution media are water, hydrochloric acid (e.g. having a concentration in the range of from about 0.001 molar to about 0.1 molar HCl), simulated gastric fluid with or without pepsin, various buffer solutions (glycine, citrate, acetate, phosphate, and borate buffers), simulated intestinal fluids with or without enzymes (e.g. 0.05 molar phosphate buffer at pH 7.5 with or without pancreatin), water contaning a surfactant, buffer solutions containing a surfactant, and aqueous alcoholic solutions (e.g. low molecular weight alcohols soluble in water typically containing 5 or less carbons to act as a cosolvent). These various parameters may be adjusted to alter solubility conditions for a given analyte. Through iterative experimentation, it is possible to empirically derive an optimal composition for a drug release medium, which may allow the experimentor to adjust the in vitro drug release rate to within a desired range. Adjustments in the solubility conditions may also alow the experimenter to discriminate in vitro between lots which behave diffently in vivo.

In a particular embodiment of the present invention a buffer solution, optionally containing a surfactant, is employed as the aqueous dissolution medium. The type of buffer solution is not particularly restricted but should be selected depending on the specific system. Buffer solutions may be selected to control the solubility of the analyte in the drug release medium, optimize the drug release profile, and optimize the degree of discrimination between important samples. Illustrative examples of buffer solutions are 0.05 molar glycine buffer at pH ranging from 2 to 3, 0.05 molar citrate buffer at pH 3, 0.05 molar acetate buffer at pH ranging from 4 to 5, 0.05 molar acetate buffer in normal saline at pH 5.5, 0.05 molar phosphate buffer at pH ranging from 6 to 8, potassium free 0.05 molar phosphate buffer at pH 6.8, 0.05 molar phsphate buffer in normal saline at pH 7.4, 0.05 molar borate buffer at pH ranging from 8 to 10. Preferred buffer solutions are 0.05 molar phosphate buffers with pH ranging from 6-7. The buffer can have any suitable molarity, for example from about 0.001M to about 0.5M, preferably from about 0.01 to about 0.1. General information for dissolution buffer preparation can be found in USP 24, pp. 2231-2240, United States Pharmacopeial Convention Inc, Jan. 1, 2000.

In another particular embodiment the aqueous dissolution medium is water, optionally containing a surfactant.

Optionally, the aqueous dissolution medium can contain a surfactant, which is another way to manipulate the solubility of the system. Typical useful surfactants are non-ionic, cationic, anionic and zwitterionic surfactants. Illustrative examples of surfactants suitable for use in the present invention are sodium dodecyl sulfate, polyoxyethylene sorbitan monoleate (Tween ₈₀™), chenodeoxycholic acid, glycocholic acid sodium salt, poly(oxyethylene)ₙ-sorbitan-monolaurate (Tween 20™), Taurocholic acid, octylphenol ethylene oxide condensate (Triton X-100™), and hexadecyltrimethylammonium bromide.

The type and amount of the surfactant will depend on the specific system of analyte, non-aqueous liquid composition and aqueous dissolution medium and can be determined by a person skilled in the art. Surfactant concentrations may be above or below the critical micelle concentration. Typical concentration ranges for the surfactant are from about 0.001% to about 1%.

The pH of the aqueous dissolution medium should be selected depending on the specific system investigated. Generally the pH of the aqueous dissolution medium will be in the range from about 1 to about 10, preferably from about 2 to about 8. It is commonly known that the pH of the aqueous dissolution medium may affect the solubility of the analyte, and is one method of manipulating the sink conditions in the experiment. By optimizing the pH of the aqueous dissolution medium, it is possible to manipulate the dissolution characteristics of some analytes. In the case of pharmaceuticals, this may make it feasible to develop a correlation between the in vitro drug release characteristics and the in vivo pharmacokinetic performance.

By optimizing the pH of the aqueous dissolution medium, in some cases the use of a surfactant is no longer necessary. As an aqueous dissolution medium, a particularly preferred system is an aqueous buffer having an optimal pH value.

By employing an aqueous dissolution medium having an optimized pH value, and eliminating the use of surfactants in the drug release medium, it may be possible to utilize a simple filtration step to isolate the analyte in solution from the rest of the emulsion mixture. This avoids the necessity for ultrafiltration, which is more complicated and time-consuming than filtration through filters having larger pore sizes. In cases in which surfactants are not employed, the experimental parameters (e.g. agitation frequency, stroke length, duration of agitation) may be chosen so that the globule (i.e. droplet or micelle) size of the emulsion is large enough so that simple filters can be used to isolate the aqueous soluble fraction of the analyte of interest, while still ensuring that the oil phase globules of the emulsion do not substantially pass the filter and detrimentally affect the determination of the amount of analyte in the aqueous dissolution medium. In cases where the emulsion micelle size is too small (e.g. less than 0.2 microns) and passes through a simple filter, ultrafiltration may be employed to isolate the analyte in solution state. Mechanical agitation parameters may also be adjusted to optimize globule size.

In one embodiment, the pH, surfactant concentration, and mechanical agitation paratmeters of the system are optimized so that the emulsion can be filtered through a filter having a pore size of about 0.2 microns (Acrodisk® syringe filter, product number 4496, Gelman Laboratory), while the oil phase globules of the emulsion substantially do not pass the filter. The physical (temperature, agitation frequency, stroke length, vessel geometry, sample size and amount of dissolution medium) and chemical parameters (pH, sink conditions, concentration of buffers, surfactants, and cosolvents) of the dissolution system can be optimized by iterative methods. A filter having the desired pore size is selected. The method of the invention is conducted using an aqueous dissolution medium having a fixed pH (e.g. pH=7). The formed emulsion is filtered through the selected filter. If the droplets of the emulsion do not substantially pass through the filter then simple filtration will suffice and the concentration of the analyte in the aqueous phase may be determined. If, however, the droplets of the emulsion do pass the filter, ultrafiltration or another suitable purification step (e.g. liquid/liquid extraction) may be needed before proceeding with quantitation of the aqueous soluble portion of the analyte. It may be determined whether the droplets substantially pass the filter by visually inspecting the filtrate. If it is clear and not hazy, then the droplets have not substantially passed the filter. Physical and chemical parameters may then be iteratively adjusted and the experiments repeated until more optimal conditions are identified. Any of the pH values classified as optimal can be used in the preferred embodiment of the present invention. The above described incremental method illustrates how the optimal pH range, surfactant type and concentration, and mechanical and thermal agitation parameters can be identified.

For measuring the disollution of an analyte in a non-aqueous liquid composition, the composition is combined with the aqueous disollution medium. The amount of composition which is combined with the aqueous dissolution medium can vary widely, depending on a variety of factors such as the nature of the composition, nature of the dissolution medium, and the amount of the dissolution medium used. Any amount that results in an analyte concentration in the aqueous phase that is detectable using a suitable analytical method may be acceptable. Thus, the ratio of the non-aqueous liquid compostion to the aqueous dissolution medium may vary widely from case to case. Typically the ratio of non-aqueous liquid composition to aqueous dissolution medium is from about 1:100 to about 1:2000, preferably from about 1:250 to about 1:1000. A further advantage of the invention is that the dissolution can be carried out with small sample volumes. In contrast to standard dissolution methods, which require volumes of aqueous dissolution medium of about 500 ml to 1000 ml, in some embodiments of the method of the invention it can be carried out using about 10 ml to about 100 ml or preferably, about 20 to about 50 ml of aqueous dissolution medium.

After the non-aqueous liquid composition and the aqueous dissolution medium are combined, the resultant mixture is agitated so that an emulsion is formed. The term “emulsion” means a dispersed system containing two or more phases in which at least two of the phases are immiscible or partially immiscible liquids.

The mixture of the non-aqueous liquid composition and the aqueous dissolution medium can be agitated in any suitable agitation apparatus. Generally the apparatus will have at least one receptacle for the mixture. A receptacle is any vessel, container, indentation or other form in which the mixture can be agitated and it should be formed so that none of the mixture is lost during agitation. The receptacle or the plurality of receptacles can either be a permanent part of the agitation apparatus or they can be separable therefrom. Typically the receptacles will be separable from the agitation apparatus so that they can be cleaned before they are reused or can be discarded. Typical receptacles include disposable EPA type vials, centrifuge tubes, test tubes, serum vials, beakers, Erlenmeyer flasks, reaction vials, or other types of containers composed of plastic, rubber, glass, metal, or treated paper. Preferred receptacles are disposable vials, either glass EPA type 40 mL vials, or glass serum vials with a rubber stopper (50 to 100 mL sizes).

Any agitation apparatus, which can be used to prepare an emulsion, can be employed. Examples of suitable agitation apparatuses are the various laboratory shakers commercially available with or without temperature control and may agitate in an orbital, linear (reciprocal), or any other fashion. Preferred agitation apparatuses are shakers in which the mixture contained in the receptacle is vigorously agitated. The shaker can move the receptacle horizontally, vertically, in a seesaw fashion or in any combination thereof. A particularly preferred shaker is a reciprocating shaker. Although they might be able to form an emulsion under special circumstances typically the non-aqueous liquid composition and the aqueous dissolution medium will not form an emulsion by simple stirring such as in a paddle assembly. In these apparatuses the non-aqueous liquid composition generally floats on the surface of the aqueous dissolution medium. Therefore, the area of contact between these two components is smaller than in the method of the invention and the dissolution rate of the analyte may be lower.

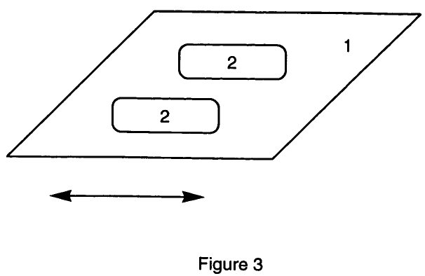

FIG. 3 is a schematic drawing of an agitation apparatus, which can be employed in the method of the present invention. It is a shaker and comprises a horizontal plate 1 to which two receptacles 2 are attached. When in action, the horizontal plate moves horizontally in the directions indicated by the arrow. Although the receptacles are shown lying on the surface of the shaker, they can also be in an upright position. This can further improve the precision and reliability of the method of the invention. If the shape of the vessel is elongated, the stroke of the shaker is preferably parallel to the elongation of the vessel as e.g. illustrated by the arrow in FIG. 3, however, it can also be in any other suitable direction.

The non-aqueous liquid composition and the aqueous dissolution medium are agitated for a predetermined time. The duration of agitation can vary greatly and will depend, for example on the amount of agitation, the analyte, the non-aqueous liquid composition, the dissolution medium, the temperature, the sensitivity of the detection method used to determine the amount of analyte and a number of other factors. Furthermore, the duration of agitation will depend on whether information on short term, medium term or long term dissolution rates or a combination of these is desired. Generally the agitation is continued until from about 1% to about 100%, preferably from about 10% to about 100%, of the total amount of analyte in the aqueous dissolution medium has been dissolved. Typically the agitation will be conducted for from about 5 minutes to about 24 hours, preferably from about 15 minutes to about 60 minutes.

During the agitation step, the mixture of the non-aqueous liquid composition and the aqueous dissolution medium can be held at any desired contacting temperature. Commonly the mixture is held at a relatively constant contacting temperature e.g. room temperature (i.e. about 22-25° C.) or at about 37° C. However, higher temperatures can be used to increase the dissolution rate and lower temperatures can be employed to slow the dissolution rate. Since the temperature of the mixture influences the dissolution rate, the same temperature should be chosen for each experiment, if the results of more than one experiment are to be compared. Within the context of the invention the “same temperature” means that the differences between the temperatures of different experiments are at most 5° C., preferably at most 2° C. Preferably the contacting temperature is room temperature (i.e. 22-25° C.).

The amount of agitation during contacting such as the shaking rate also influences the dissolution rate of the analyte and the optimal conditions should be determined based on various factors such as the size and shape of the agitation vessel, the non-aqueous liquid composition, and the aqueous dissolution medium. Typically the number of cycles will be in the range from about 100 to about 500, preferably from about 100 to about 300. The stroke length is preferably from about 0.5 inch to about 2 inches, more preferably from about 0.75 inch to about 1.5 inch.

After the mixture of the non-aqueous liquid composition and the aqueous dissolution medium has been agititated for a predetermined amount of time, the amount of analyte in the aqueous dissolution medium is determined. With some detection methods the amount of analyte can be determined while the aqueous dissolution medium remains in the dissolution testing apparatus. Typically, however, at least part of the aqueous dissolution medium is removed from the dissolution testing apparatus, e.g. by means of a syringe or a permanent sampling tube. Although it is possible to use all of the aqueous dissolution medium for the analysis and this might be necessary with some detection methods, in other cases only part of the aqueous dissolution medium will be employed. The size of the sample removed for determining the amount of analyte will depend on a variety of factors, particularly on the employed detection method, and can be from about 0.1 mL to about 25 mL, preferably from about 0.5 mL to about 15 mL.

If desired, the sample of the aqueous dissolution medium, which is to be used for determining the amount of analyte, can be filtered or centrifuged after it has been removed from the dissolution testing apparatus. This may be done to isolate the aqueous phase from oil and solid phases, thus removing particles or droplets of emulsion containing analyte from the aqueous phase, which might interfere with the determination of the analyte and confound the measurement. Filtration can be achieved by any suitable means such as filtering through a filter having an average pore size of from about 0.1 to about 50 microns, preferably from about 0.1 to about 0.3 microns. In some cases ultrafiltration might be necessary. Then filters having a pore size of from about 0.001 micron to about 0.1 micron, preferably from about 0.001 to about 0.01 would be more appropriate. There are numerous examples of suitable filter materials known in the art. These filters are, for example commercially available under the trade designations Acrodisk® from Gelman Laboratory and Centriprep 50® from Millipore Corporation.

After the optional filtering step, the amount of analyte in the aqueous dissolution medium is determined. Any analytical method suitable for determining the amount of analyte can be employed. The choice of the analytical method will depend on a variety of parameters including the nature of the analyte, its concentration range, the dissolution medium, and also which methods are available in the laboratory. Illustrative examples of analytical methods are separation techniques (e.g. high performance liquid chromatograhy, liquid chromatography, thin layer chromatography, capillary electrophoresis, gas chromatography), photometric and spectrophotometric techniques (e.g. ultraviolet-visible (UV-Vis), Fourier transform infrared (FTIR), atomic absorption (AA), atomic emission (AE), mass spectrometry (MS)). Chromatographic methods, in particular gas chromatography (GC) and high performance liquid chromatography (HPLC), are preferred. Examples of suitable chromatographic methods are reverse phase high performance liquid chromatography (RP-HPLC), normal phase high performance liquid chromatography (NP-HPLC), incorporating any of a variety of detection techniques known in the art. Examples of detection techniques which may be used in conjunction with a suitable chromatographic method include, UV-Vis, index of refraction, mass spectrometry and light scattering detection. Flow injection analysis (FIA) with UV-Vis detection can also be employed as an analytical method. FIA is particularly suitable when a high throughput of samples is needed, such as is the case when performing in-process characterization of a manufacturing system in real time.

The method of the invention has been explained supra with respect to an embodiment in which the amount of analyte dissolved at a single predetermined point of time is determined. In many cases, it is of interest to monitor the dissolution rate over a period of time to determine whether the analyte is released at a constant rate or if the rate varies with time (e.g. a large amount at the beginning of the dissolution testing and then lesser amounts later on). In these cases, it is possible to use a sufficiently large receptacle, to remove two or more samples therefrom at different predetermined times and to analyze these samples individually. It is also possible to prepare two or more identical experiments and to agitate them under identical conditions with the exception that the duration of agitation is varied. In this case, as shown in FIG. 3 the individual mixtures can be agitated simultaneously using the same agitation apparatus. It is also possible to agitate the mixtures one after the other or by using different apparatuses. The aqueous dissolution medium sampled at the various points of time from these separate mixtures is analyzed individually. The results can then be used to determine the time-dependent profile of the dissolution rate.

Using the method of the invention it is now possible to reliably and accurately measure the dissolution rate of an analyte in a non-aqueous liquid composition. In pharmaceutical applications, the results obtained with the in vitro method of the invention may correlate well with the results of in vivo pharmacokinetic studies. Therefore, it can be used as a rugged and reliable method in quality control during the manufacture of pharmaceuticals to ensure adequate bioperformance and lot consistency. Since the method is simple, cheap and fast, it can also be used with advantage in the development of pharmaceuticals and their dosage forms. The method of the invention is useful for determining the dissolution rate of drugs, which have a slow dissolution rate, or sustained release dosage forms, since it is quicker than conventional methods. The method is particularly useful for real time monitoring of manufacturing processes intended to attenuate drug release (impart sustained release characteristics) in pharmaceutical dosage forms.

The following examples are presented to illustrate the invention. However, they should not be construed as limiting. Unless otherwise mentioned all percentages, ratios, parts, etc. referred to in the examples are by weight.

### EXAMPLES

Precision:

The precision of the methods of the present invention can be determined by calculating the relative standard deviation (RSD) of repeat measurements. A lower relative standard deviation value indicates a higher precision. Typically, the relative standard deviation is determined by measuring the dissolution rate of an analyte under identical conditions with replication greater than two. The relative standard deviation is then calculated according to the following formula:

$$RSD = \frac{s . d .}{\overline{X}} \times 100$$

where s.d. is the standard deviation which is defined as:

$$s . d . = \sqrt{\frac{{\sum }^{} {( X - \overline{X} )}^{2}}{N - 1}} ;$$

X is the individual result; N is the number of data points; and \overline{X} is the mean.

The relative standard deviation obtained with the method of the invention can be lower than 1%.

Accuracy:

The accuracy of the methods of the present invention can be determined by measuring the transfer of an analyte from a non-aqueous liquid composition to an aqueous medium where the non-aqueous liquid composition is spiked with a known amount of analyte. The spiked non-aqueous liquid composition is equilibrated with the aqueous drug release medium by shaking or stirring, after which the amount of analyte in the aqueous dissolution medium is determined. The concentration of analyte which transferred to the aqueous medium is then compared to the concentration which would result, in theory, if 100% of the analyte had transferred. (e.g. under the assumptions that no pipetting, weighing errors or losses occur, that 100% of the analyte has dissolved and that 100% of the analyte is detected). Methods of the invention are accurate within the range of from about 70% to about 100%, preferably from about 90% to about 100%.

General Dissolution Procedure

| Equipment: | |
|---|---|
| Platform Shaker: | Reciprocating shaker model 5850 |
| | commercially available from |
| | Eberbach, nominal stroke length |
| | approximately 2.54 cm, frequency |
| | 200 cycles/minute. The vials were |
| | in a horizontal position and were |
| | aligned parallel to the stroke |
| | direction as indicated in FIG. 3. |
| | Maintain platform shaker in a |
| | suitably controlled temperature |
| | environment (e.g. 22° C.). |
| Vials: | 40 mL (EPA type) Teflon lined |
| | screw cap. Commercially available |
| | from Qorpak (part number 7588T). |
| Plastic Syringes: | BD Disposable 10 mL plastic |
| | syringes or equivalent. |
| Filters: | Acrodisk 0.2 micron (part number |
| | 4496) |

Procedure:

Dispense an appropriate amount (e.g. 30-70 mg) of the composition for dissolution testing into an empty 40 mL vial. Equilibrate at appropriate temperature (e.g. 22° C.). Equilibrate the dissolution medium at an appropriate temperature (e.g. 22° C.). Add an appropriate volume (e.g. 30 mL) of dissolution medium into the vial containing the sample for dissolution testing. Repeat for all samples. Complete this process within about 2-3 minutes. Start agitation.

At predetermined time points, remove samples for quantitative analysis. Filter if necessary. Proceed to quantitative analysis.

General Quantitative Analytical Procedure

Unless otherwise mentioned the following general procedure was followed.

| | |
|---|---|
| Apparatus: | HPLC capable of isocratic operation |
| | (e.g. Agilent 1100 commercially |
| | available from Agilent Technologies). |
| Detector: | UV-Vis Detector at 254 nm (e.g. |
| | Diode array detector, detection |
| | wavelength: 254 nm, commercially |
| | available from Agilent Technologies). |
| Column: | Waters Symmetry C8, 3.9 × 50 mm, |
| | commercially available from Waters |
| | Corporation. |
| Injection volume: | 10 μl |
| Flow rate: | 1-2 ml/min |
| Pressure: | 3000 psi |
| Mobile phase: | 3.85 g ammonium acetate, 13.5 ml |
| | of 40% tetrabutylammonium hydroxide |
| | were dissolved in Milli-Q water to |
| | give a total volume of 700 ml. The |
| | pH was adjusted to 6.7 ± 0.1 with |
| | glacial acetic acid. Then the solution |
| | was filtered through a 0.45 μm |
| | membrane filter. After filtration |
| | 200 ml methanol and 110 ml |
| | tetrahydrofuran were added and the |
| | mixture was sonicated under vacuum to |
| | degas it. |

Calculation of the Released Amount of Analyte

The amount of released analyte (e.g. Ceftiofur) at each point in time can be calculated according to the following formula.

$$\frac{mg Analyte released}{gram} = \frac{( Wstd * P )}{Rstd} * \frac{DISVOL}{WSVOL} * \frac{Rsam}{Wsam} * \frac{1000}{1}$$

where,

Wstd=Weight of the standard preparation, in mg

P=Purity of the reference standard as Ceftiofur Free Acid

Rstd=Peak area of Standard Preparation

DISVOL=Volume of dissolution fluid, in mL (30)

WSVOL=Volume of working standard, in mL (10)

Rsam=Peak area of Sample Preparation

Wsam=Weight of sample suspension in mg

1000=Conversion of sample weight from mg to gram

### Example 1

Two CCFA supension samples were tested using the methods described below.

The nonaqueous vehicle was prepared by pumping cottonseed oil into a jacketed vessel and heating to 115° C. Phospholipon 90H was added (0.05% by weight) (available from American Lecithin Co.) and mixed. The solution was cooled to 45° C. Sorbitan monooleate was added (0.15% by weight) and mixed. CCFA was added at 100 mg/mL and mixed through a triblender until the suspension was homogeneous. The suspension was recirculated through the triblender, with tank agitator running and screened. The resultant suspension was filled in sterile vials, stoppered and oversealed. The sealed vials were sterilized using gamma irradiation. The lots were labeled 40,700 and 40,620.

An aqueous dissolution medium was prepared by dissolving 1% Tween 80 surfactant (available from Sigma-Aldrich), in 0.05 molar pH 6.0 phosphate buffer. The stock pH 6.0 phosphate buffer was prepared by adding 21.8 grams potassium phosphate monobasic and 3.48 grams potassium phosphate dibasic to deionized water and diluting to 200 mL. The 0.05 molar pH 6.0 phosphate buffer was prepared by diluting 50 mL of stock buffer to 900 mL with deionized water.

Between 65 and 85 miligrams of CCFA suspension Lot No. 40,700 and 30 ml aqueous dissolution medium was introduced into a 40 ml EPA type glass vial with a Teflon®-lined screw cap (available from Qorpak). A total of nine identical vials were prepared. The process was repeated using a second lot of CCFA suspension Lot No. 40,620 to prepare nine additional vials. The vials were placed horizontally onto a reciprocating shaker (Eberbach Model 5850, stroke length=17/16 inch, shaking frequency=200 cycles per minute) and agitation was started. After 15 minutes of agitation, the first three vials of each lot were removed from the shaker. A 15 milliliter sample was taken from each vial and ultrafiltered using a Centriprep ultrafiltration device (Centriprep 50 ultrafiltration device, available from Amicon or Millipore Corporation). The amount of CCFA in the filtrate was determined using the general analytical process described above. The process was repeated at 60 minutes and 120 minutes of agitation; the remaining vials of each lot were respectively removed and treated in a similar fashion.

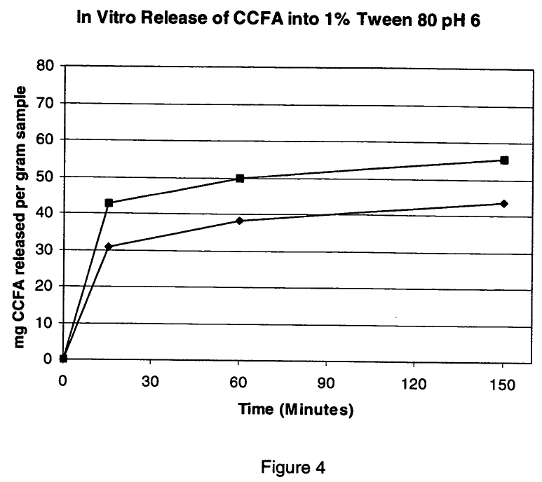

The obtained results are shown in FIG. 4 (Lot 40,620 data represented with diamonds, Lot 40,700 data represented with squares). The measured dissolution rate correlated to the pharmacokinietic performance that was previously observed in in vivo experiments.

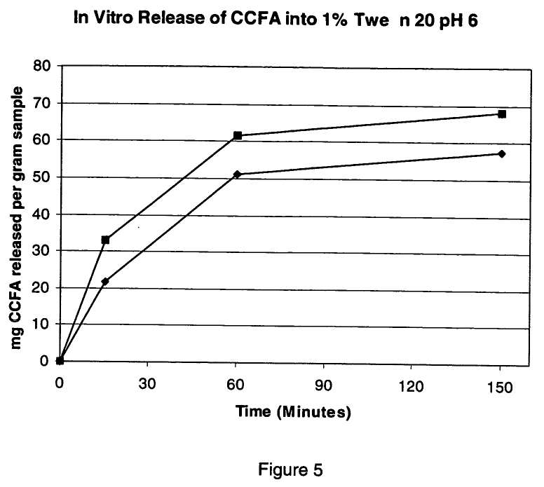

The experiments were repeated using 1% Tween 20 as a surfactant instead of 1% Tween 80. The results are shown in FIG. 5 (Lot 40,620 data represented with diamonds, Lot 40,700 data represented with squares). The measured dissolution rate correlated to the pharmacokinietic performance that was previously observed in in vivo experiments.

### Example 2

This example illustrates the correlation between the in vitro results obtained with the method of the present invention and the in vivo results obtained in pharmacokinetic studies.

Three lots (40,620; 40,700; JMS-144F) of CCFA S. Suspension which exhibited different in vivo pharmacokinetic performance were used in this evaluation. Although each lot had the same composition, and was manufactured using the same process described above in Example 1 (except that JMS-144F was not sterilized by treatment with gamma irradiation), the lots exhibited different in vivo pharmacokinetic performance. The differences in pharmacokinetic performance were evidenced by differences in the duration of the sustained release effect, which is given by the number of hours which ceftiofur was detected in the blood stream of the animals. The analytical detection limit for ceftiofur is 0.2 microgram ceftiofur per mL of plasma. The duration of the sustained release effect is commonly referred to as “time above 0.2 mcg/mL”.

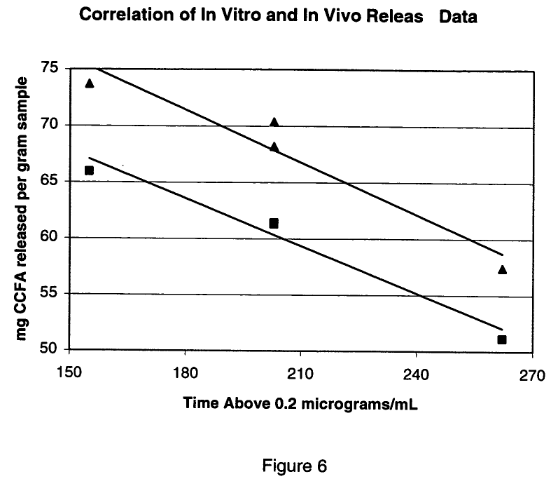

The three lots were tested with the method described in this invention. The correlation of in vitro drug release results with time above 0.2 mcg/mL is shown in FIG. 6. In vitro results are given for 60 (data points represented by squares) and 150 minute (data points represented by triangles) time points. The solid lines are the best least squares fit for the data and are included to illustrate the inverse correlation between amount released, in vitro, and the duration of the sustained release effect observed in vivo. Lots which released more CCFA at a given time in vitro, had shorter duration of sustained release in vivo.

### Example 3

This example illustrates that by optimizing the pH of the aqueous dissolution medium, one can eliminate the use of surfactants in the drug release medium and also eliminate the necessity for ultrafiltration.

In this example, CCFA was employed as the analyte. The solubility of CCFA varies as a function of pH. Below pH 5, CCFA is relatively insoluble; as pH increases, the solubility of CCFA increases.

This drug release experiment employed two different drug release media: (1) 1% Tween 20 in pH 6.0 phosphate buffer, and (2) pH 6.5 phosphate buffer containing no exogenous surfactants. The buffers were prepared in the following manner:

0.05 molar pH 6.0 phosphate buffer was prepared by adding 21.8 grams potassium phosphate monobasic and 3.48 grams potassium phosphate dibasic to deionized water and diluting to 200 mL, then diluting again by a factor of 18 with deionized water (i.e. 50 mL diluted to 900 mL with deionized water).

0.05 molar pH 6.5 phosphate buffer was prepared by adding 31.98 grams of potassium phosphate monobasic and 15.39 grams potassium phosphate dibasic to deionized water and diluting to 1000 mL, then diluting again by a factor of 10 with deionized water (i.e. 100 mL of stock buffer to 1000 mL with deionized water).

Three lots of CCFA S. Suspension (40,620; 40,700; JMS-111) which exhibited different in vivo pharmacokinetic performance were used in this evaluation. Although each lot had the same composition, and was manfactured using the same process described above in Example 1 (except that lot JMS-111 was not sterilized by treatment with gamma irradiation), the lots exhibited different in vitro drug release rates when tested with the initial drug release medium (1% Tween 20 in pH 6.0 phosphate buffer). The variable in vitro release between lots is reflected in the varying amounts of CCFA released in 60 minutes (TABLE 1).

Drug release medium was prepared consisting of pH 6.5 phosphate buffer without exogenous surfactant, and the in vitro drug release measurements were repeated. By raising the pH from 6.0 to 6.5, and removing Tween 20 from the drug release medium, it was found that the rank order corelation of in vitro release rates for the three lots remained the same, and that oil phase globules of the emulsion were considerably larger, and could be removed by simple filtration. Thus the ultrafiltration purification step could be replaced with simple filtration. Additionally, it was found that the in vitro release rates for all three samples were faster in the pH 6.5 medium. Therefore, the assay time could be shortened from 60 minutes to 30 minutes. Finally, it was found that adequate absolute resolution between the three CCFA lots could be achieved, and that the faster (30 minutes vs. 60 minutes) and simpler (elimination of Tween 20, and simple vs. ultrafiltration) procedure could adequately discriminate between lots with various in vitro release rates. Absolute resolution was defined as the difference between the amounts of CCFA released for lots within a drug release medium at a time point chosen specifically for that medium.

TABLE 1

| | | | | Absolute Resolution | |
|---|---|---|---|---|---|
| drug | | | | between Lots | |
| release | CCFA Released by Lot | | | (40,700- | JMS111- |
| medium | 40620 | 40700 | JMS111 | 40,620) | 40620) |
| pH = 6.0 | 48.2 | 60.4 | 29.4 | 12.2 | −18.8 |
| with 1% | mg in | mg in | mg in | | |
| Tween 20 | 60 | 60 | 60 | | |
| | minutes | minutes | minutes | | |
| pH = 6.5 | 71.5 | 87.8 | 47.9 | 16.3 | −23.6 |
| without | mg in | mg in | mg in | | |
| exogenous | 30 | 30 | 30 | | |
| surfactant | minutes | minutes | minutes | | |

### Example 4

Two different non-aqueous bases for CCFA suspensions were prepared and each was spiked with CCFA. Spikes were prepared in triplicate at 2 nominal concentration levels. The exact amount of CCFA added was recorded.

The non-aqueous base of Base-1 was 100% coconut oil (available as Miglyol 812), while the non-aqueous base of Base-2 was a 1:1 (v:v) mixture of coconut oil (available as Miglyol 812 from HulsAmerica) and cottonseed oil (available from Welch, Home & Clark Company).

The CCFA spiked Base mixtures were prepared by precisely adding known amounts of drug to approximately 65 mg of Base (1 or 2) in 40 mL EPA vials and mixing. Following preparation of the base mixtures, the samples were extracted with dissolution medium by shaking on a reciprocating shaker for four hours. The samples were filtered, and the concentration of CCFA in the filtrate was determined by HPLC. Results are summarized in Table 2. The results ranged from 98.4 to 100.1% CCFA recovered.

TABLE 2
The recovery of CCFA in the test solutions.

| | Amount | Amount |
|---|---|---|
| non-aqueous | added | recovered |
| base | [mg/ml] | [%] |
| Base-1 | 50.2 | 99.655 |
| | 50.4 | 99.266 |
| | 50.1 | 100.082 |
| | 100.9 | 98.367 |
| | 100.1 | 99.162 |
| | 100.3 | 99.243 |
| Base-2 | 50.2 | 99.455 |
| | 50.4 | 99.565 |
| | 50.5 | 99.807 |
| | 100.4 | 99.094 |
| | 100.8 | 99.186 |
| | 100.4 | 99.531 |

What is claimed is:

1. A method of characterizing the transfer of an analyte from a non-aqueous liquid composition to an aqueous medium, comprising the steps of:
(a) providing a non-aqueous liquid composition comprising an analyte and a non-aqueous base;
(b) combining the non-aqueous liquid composition with an aqueous dissolution medium;
(c) agitating the non-aqueous liquid composition and the aqueous dissolution medium to form an emulsion; and
(d) determining the amount of the analyte in the aqueous dissolution medium.

2. The method of claim 1, wherein in step (d) the amount of analyte in the aqueous dissolution medium is determined at more than one point in time.

3. The method of claim 1, further including a step of passing the aqueous dissolution medium, which is to be used for determining the amount of analyte in the aqueous dissolution medium, through a filter before determining the amount of analyte in the aqueous dissolution medium.

4. The method of claim 3, wherein the pore size of the filter ranges from about 0.1 to about 50 microns.

5. The method of claim 1, wherein the non-aqueous liquid composition is a pharmaceutical composition.

6. The method of claim 5, wherein the analyte is a pharmaceutically active component in the pharmaceutical composition.

7. The method of claim 5, wherein the pharmaceutical composition is a sustained release dosage form.

8. The method of claim 5, wherein the pharmaceutical composition further contains pharmaceutically acceptable components.

9. The method of claim 1, wherein the analyte is selected from ACE inhibitors; □-adrenergic agonists; □-adrenergic agonists; □-adrenergic blockers; □-adrenergic blockers (beta blockers); alcohol deterrents; aldose reductase inhibitors; aldosterone antagonists; amino acids; anabolics; analgesics (both narcotic and non-narcotic); anesthetics; anorexics; antacids; anthelmintics; antiacne agents; antiallergics; antiandrogens; antianginal agents; antianxiety agents; antiarrythmics; antiasthmatics; antibacterial agents and antibiotics; antialopecia and antibaldness agents; antiamebics; antibodies; anticholinergic drugs; anticoagulants and blood thinners; anticolitis drugs; anticonvulsants; anticystitis drugs; antidepressants; antidiabetic agents; antidiarrheals; antidiuretics; antidotes; antiemetics; antiestrogens; antiflatulents; antifungal agents; antigens; antiglaucoma agents; antihistaminics; antihyperactives; antihyperlipoproteinemics; antihypertensives; antihyperthyroid agents; antihypotensives; antihypothyroid agents; anti-infectives; anti-inflammatories (both steroidal and nonsteroidal); antimalarial agents; antimigraine agents; antineoplastics; antiobesity agents; antiparkinsonian agents and antidyskinetics; antipneumonia agents; antiprotozoal agents; antipruritics; antipsoriatics; antipsychotics; antipyretics; antirheumatics; antisecretory agents; anti-shock medications; antispasmodics; antithrombotics; antitumor agents; antitussives; antiulceratives; antiviral agents; anxiolytics; bactericidins; bone densifiers; bronchodilators; calcium channel blockers; carbonic anhydrase inhibitors; cardiotonics and heart stimulants; chemotherapeutics; choleretics; cholinergics; chronic fatigue syndrome medications; CNS stimulants; coagulants; contraceptives; cystic fibrosis medications; decongestants; diuretics; dopamine receptor agonists; dopamine receptor antagonists; enzymes; estrogens; expectorants; gastric hyperactivity medications; glucocorticoids; hemostatics; HMG CoA reductase inhibitors; hormones; hypnotics; immunomodulators; immunosuppressants; laxatives; medicaments for oral and periodontal diseases; miotics; monoamine oxidase inhibitors; mucolytics; multiple sclerosis medications; muscle relaxants; mydriatics; narcotic antagonists; NMDA receptor antagonists; oligonucleotides; ophthalmic drugs; oxytocics; peptides, polypeptides and proteins; polysaccharides; progestogens; prostaglandins; protease inhibitors; respiratory stimulants; sedatives; serotonin uptake inhibitors; sex hormones including androgens; smoking cessation drugs; smooth muscle relaxants; smooth muscle stimulants; thrombolytics; tranquilizers; urinary acidifiers; urinary incontinence medications; vasodilators; vasoprotectants; and combinations thereof

10. The method of claim 1, wherein the analyte is a cephalosporin.

11. The method of claim 1, wherein the analyte is ceftiofur, a pharmaceutically acceptable salt or derivative thereof.

12. The method of claim 1, wherein the non-aqueous base is a lipid.

13. The method of claim 12, wherein the non-aqueous base is an oil.

14. The method of claim 13, wherein the oil is selected from the group consisting of canola oil, coconut oil, corn oil, peanut oil, sesame oil, olive oil, palm oil, safflower oil, soybean oil, cottonseed oil, rapeseed oil, sunflower oil and mixtures thereof.

15. The method of claim 14, wherein the oil is cottonseed oil.

16. The method of claim 1, wherein the non-aqueous liquid composition is a suspension, solution or emulsion.

17. The method of claim 1, wherein the non-aqueous liquid composition is a suspension.

18. The method of claim 1, wherein the agitation is conducted until from about 10% to about 100% of the total amount of analyte, which was initially present in the non-aqueous liquid composition, has been dissolved in the aqueous dissolution medium.

19. The method of claim 1, wherein the aqueous dissolution medium comprises a buffer.

20. The method of claim 19, wherein the buffer is selected from the group consisting of glycine buffer, citrate buffer, acetate buffer, phosphate buffer, and borate buffer.

21. The method of claim 20, wherein the buffer has an optimal pH.

22. The method of claim 1, wherein the aqueous dissolution medium has an optimal pH.

24. The method of claim 1, wherein the aqueous dissolution medium is free of surfactant.

25. The method of claim 21, wherein the aqueous dissolution medium is free of surfactant.

26. The method of claim 22, wherein the aqueous dissolution medium is free of surfactant.

27. The method of claim 1, wherein the aqueous dissolution medium comprises a surfactant.

28. The method of claim 1, wherein the ratio of non-aqueous liquid composition to aqueous dissolution medium by weight is from about 1:100 to about 1:2000.

29. The method of claim 1, wherein step (c) is carried out on a shaker.

30. The method of claim 29, wherein the shaker is a reciprocating shaker.

31. The method of claim 29, wherein the shaker has a stroke rate of from about 50 to about 400 cycles per minute.

32. The method of claims 1 or 29 wherein in step (c) the non-aqueous liquid composition and the aqueous dissolution medium are contained in a 40 mL EPA type vial or a 50-100 mL serum type vial.

33. The method of claim 8, wherein the pharmaceutically acceptable component is selected from the group consisting of excipients, additives, suspending agents, preservatives, wetting agents, thickeners, buffers, flocculating agents, flavoring agents, sweeteners, colorants and fragrances.

34. The method of claim 10, wherein the cephalosporin is selected from the group consisting of ceftiofur, cefepime, cefixime, cefoperazone, cefotaxime, cefpodoxime, ceftazidime, ceftizoxime, ceftriaxone, moxalactam, and pharmaceutically acceptable salts and derivatives thereof.

35. The method of claim 1 wherein the aqueous dissolution medium is selected from water, a hydrochloric acid solution, a simulated gastric fluid, a buffer solution, a simulated intestinal fluid, water containing a surfactant, a buffer solution containing a surfactant, and an aqueous alcoholic solution.

36. The method of claim 1 wherein the non-aqueous liquid composition is a suspension, solution, or dispersion.

37. The method of claim 1 provided that the non-aqueous liquid composition is not an emulsion.

38. A method of predicting the in vivo performance of a non-aqueous pharmaceutical composition comprising an analyte, said method comprising the step of characterizing the transfer of the analyte from the composition to an aqueous medium according to the method of claim 1.

39. A method of commercially producing and releasing a non-aqueous pharmaceutical composition comprising an analyte for public use, comprising the steps of:
(a) preparing the non-aqueous pharmaceutical composition;
(b) characterizing the transfer of the analyte from the composition to an aqueous medium according to the method of claim 1; and
(c) confirming that the result from step (b) falls within desired standards.
